(19) United States
(12) Patent Application Publication
(10) Pub. No.: US 2014/0128739 A1
(43) Pub. Date: May 8, 2014

(54) ULTRASOUND IMAGING SYSTEM AND METHOD

(71) Applicant: GENERAL ELECTRIC COMPANY, Schenectady, NY (US)

(72) Inventors: Subin Baby Sarojam Sundaran, Bangalore (IN); Halmann Menachem, Wauwatosa, WI (US)

(73) Assignee: GENERAL ELECTRIC COMPANY, Schenectady, NY (US)

(21) Appl. No.: 13/723,828

(22) Filed: Dec. 21, 2012

(30) Foreign Application Priority Data
Nov. 7, 2012 (IN) 4659/CHE/2012

(51) Int. Cl. A61B 8/00; A61B 8/08; A61B 8/06; A61B 8/14

(52) U.S. Cl. CPC A61B 8/4254; A61B 8/54; A61B 8/4444; A61B 8/461; A61B 8/14; A61B 8/481; A61B 8/06; A61B 8/488; A61B 8/486; A61B 8/485; A61B 8/483
USPC 600/459

(57) ABSTRACT

An ultrasound imaging system and method includes performing a gesture with a probe and detecting the gesture based on data from a motion sensing system in the probe. The motion sensing system includes at least one sensor selected from the group of an accelerometer, a gyro sensor and a magnetic sensor. The ultrasound imaging system and method also includes performing a control operation based on the detected gesture.

### FIELD OF THE INVENTION

This disclosure relates generally to an ultrasound imaging system and a method for performing a control operation based on a gestured performed with a probe.

### BACKGROUND OF THE INVENTION

Conventional hand-held ultrasound imaging systems typically include a probe and a scan system. The probe contains one or more transducer elements that are used to transmit and receive ultrasound energy. The controls used to control the hand-held ultrasound imaging system are typically located on the scan system. For example, the user may control functions such as selecting a mode, adjusting a parameter, or selecting a measurement point based on control inputs applied to the scan system. Some conventional hand-held ultrasound imaging systems use touch screens as part or all of the user interface. Other conventional hand-held ultrasound imaging systems include a plurality of hard keys on the scan system to control imaging operations. When using a hand-held ultrasound imaging system, both of the user's hands are typically occupied. For example, a user would typically hold the probe in one hand while holding the scan system in their other hand. Since both hands are occupied while scanning with a typical hand-held ultrasound imaging system, it can be difficult for the user to perform various control operations. In additional, with a conventional hand-held ultrasound imaging system, it can be especially difficult for the user to perform specific measurements or other operations that require the precise placement of one or more points.

For these and other reasons an improved ultrasound imaging system and an improved method for controlling an ultrasound imaging system are desired.

### BRIEF DESCRIPTION OF THE INVENTION

The above-mentioned shortcomings, disadvantages and problems are addressed herein which will be understood by reading and understanding the following specification.

In an embodiment, a method of controlling an ultrasound imaging system includes performing a gesture with a probe and detecting the gesture based on data from a motion sensing system in the probe. The motion sensing system includes at least one sensor selected from the group consisting of an accelerometer, a gyro sensor, and a magnetic sensor. The method includes performing a control operation based on the detected gesture.

In an embodiment, a method of controlling an ultrasound imaging system includes inputting a command to select a measurement mode, displaying a graphical indicator on a display device, and performing a gesture with a probe. The method includes detecting the gesture based on data from a motion sensing system in the probe. The motion sensing system includes at least one sensor selected from a group consisting of an accelerometer, a gyro sensor, and a magnetic sensor. The method includes repositioning the graphical indicator based on the detected gesture. The method includes selecting a position indicated by the graphical indicator after repositioning the graphical indicator and performing a measurement using the selected position.

In another embodiment, an ultrasound imaging system includes a probe. The probe includes a housing, at least one transducer element disposed in the housing, and a motion sensing system either attached to the housing or disposed in the housing. The system also includes a scan system in communication with the probe. The scan system includes a display device, a processor configured to receive data from the motion sensing system and to interpret the data as a gesture. The processor is configured to perform a control operation based on the gesture.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a schematic representation of an ultrasound imaging system in accordance with an embodiment;

FIG. 3 is a schematic representation of a probe in accordance with an embodiment;

FIG. 4 is a schematic representation of a probe in accordance with an embodiment;

FIG. 5 is a schematic representation of a probe in accordance with an embodiment;

FIG. 6 is a schematic representation of a hand-held ultrasound imaging system in accordance with an embodiment;

FIG. 7 is schematic representation of a probe overlaid on a Cartesian coordinate system in accordance with an embodiment;

FIG. 8 is schematic representation of a scan acquisition pattern in accordance with an embodiment;

FIG. 9 is schematic representation of a scan acquisition pattern in accordance with an embodiment;

FIG. 10 is schematic representation of a scan acquisition pattern in accordance with an embodiment; and

FIG. 11 is schematic representation of a scan acquisition pattern in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

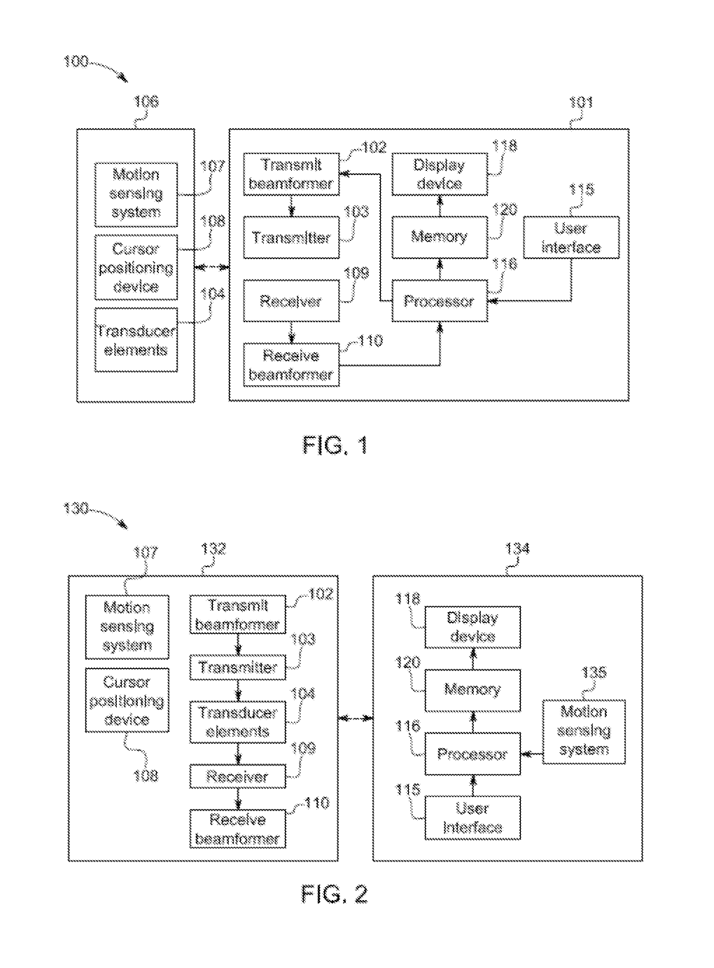

FIG. 1 is a schematic diagram of an ultrasound imaging system 100 in accordance with an embodiment. The ultrasound imaging system includes a scan system 101. According to an exemplary embodiment, the scan system 101 may be a hand-held device. For example, the scan system 101 may be similar in size to a smartphone, a personal digital assistant or a tablet. According to other embodiments, the scan system 101 may be configured as a laptop or cart-based system. The ultrasound imaging system 100 includes a transmit beamformer 102 and a transmitter 103 that drive transducer elements 104 within a probe 106 to emit pulsed ultrasonic signals into a body (not shown). The probe 106 also includes a motion sensing system 107 and a cursor positioning device 108 in accordance with an embodiment. The motion sensing system 107 may include one or more of the following sensors: a gyro sensor, an accelerometer, and a magnetic sensor. The motion sensing system 107 is adapted to determine the position and orientation of the ultrasound probe 106, preferably in real-time, as a clinician is manipulating the probe 106. For purposes of this disclosure, the term “real-time” is defined to include an operation or procedure that is performed without any intentional delay. According to other embodiments, the probe 106 may not include the cursor positioning device 108. The scan system 101 is in communication with the probe 106. The scan system 101 may be physically connected to the probe 106, or the scan system 101 may be in communication with the probe 106 via a wireless communication technique. Still referring to FIG. 1, the pulsed ultrasonic signals are back-scattered from structures in the body, like blood cells or muscular tissue, to produce echoes that return to the elements 104. The echoes are converted into electrical signals, or ultrasound data, by the elements 104 and the electrical signals are received by a receiver 109. The electrical signals representing the received echoes are passed through a receive beamformer 110 that outputs ultrasound data. According to some embodiments, the probe 106 may contain electronic circuitry to do all or part of the transmit and/or the receive beamforming. For example, all or part of the transmit beamformer 102, the transmitter 103, the receiver 109 and the receive beamformer 110 may be situated within the probe 106. The terms “scan” or “scanning” may also be used in this disclosure to refer to acquiring data through the process of transmitting and receiving ultrasonic signals. The terms “data” or “ultrasound data” may be used in this disclosure to refer to either one or more datasets acquired with an ultrasound imaging system. A user interface 115 may be used to control operation of the ultrasound imaging system 100, including, to control the input of patient data, to change a scanning or display parameter, and the like. The user interface 115 may include one or more of the following: a rotary knob, a keyboard, a mouse, a trackball, a track pad, and a touch screen.

The ultrasound imaging system 100 also includes a processor 116 to control the transmit beamformer 102, the transmitter 103, the receiver 109 and the receive beamformer 110. The processor 116 is in communication with the probe 106. The processor 116 may control the probe 106 to acquire ultrasound data. The processor 116 controls which of the elements 104 are active and the shape of a beam emitted from the probe 106. The processor 116 is also in communication with a display device 118, and the processor 116 may process the data into images for display on the display device 118. According to other embodiments, part or all of the display device 118 may be used as the user interface. For example, some or all of the display device 118 may be enabled as a touch screen or a multi-touch screen. For purposes of this disclosure, the phrase “in communication” may be defined to include both wired and wireless connections. The processor 116 may include a central processor (CPU) according to an embodiment. According to other embodiments, the processor 116 may include other electronic components capable of carrying out processing functions, such as a digital signal processor, a field-programmable gate array (FPGA) or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the RF data and generates raw data. In another embodiment the demodulation can be carried out earlier in the processing chain. The processor 116 may be adapted to perform one or more processing operations according to a plurality of selectable ultrasound modalities on the data. The data may be processed in real-time during a scanning session as the echo signals are received. Some embodiments of the invention may include multiple processors (not shown) to handle the processing tasks. For example, a first processor may be utilized to demodulate and decimate the RF signal while a second processor may be used to further process the data prior to displaying an image. It should be appreciated that other embodiments may use a different arrangement of processors.

The ultrasound imaging system 100 may continuously acquire data at a frame rate of, for example, 10 Hz to 50 Hz. Images generated from the data may be refreshed at a similar rate. Other embodiments may acquire and display data at different rates. A memory 120 is included for storing processed frames of acquired data. In an exemplary embodiment, the memory 120 is of sufficient capacity to store at least several seconds worth of frames of ultrasound data. The frames of data are stored in a manner to facilitate retrieval thereof according to its order or time of acquisition. The memory 120 may comprise any known data storage medium. According to an embodiment, the memory 120 may be a ring buffer or circular buffer.

Optionally, embodiments of the present invention may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from coordinate beam space to display space coordinates. A video processor module may be provided that reads the image frames from a memory and displays the image frames in real time while a procedure is being carried out on a patient. A video processor module may store the image frames in an image memory, from which the images are read and displayed.

FIG. 2 is a schematic representation of an ultrasound imaging system 130 in accordance with another embodiment. The ultrasound imaging system 130 includes the same components as the ultrasound imaging system 100, but the components are arranged differently. Common reference numbers are used to identify identical components within this disclosure. A probe 132 includes the transmit beamformer 102, the transmitter 103, the receiver 109 and the beamformer 110 in addition to the motion sensing system 107, the cursor positioning device 108, and the transducer elements 104. The probe 132 is in communication with a scan system 134. The probe 132 and the scan system 134 may be physically connected, such as through a cable, or they may be in communication through a wireless technique. The elements in the ultrasound imaging system 130 may interact with each other in the same manner as that previously described for the ultrasound imaging system 100 (shown in FIG. 1). The processor 116 may control the transmit beamformer 102 and the transmitter 103, which in turn, control the firing of the transducer elements 104. The motion sensing system 107 and the cursor positioning device 108 may also be in communication with the processor 116. Additionally, the receiver 109 and the receive beamformer 110 may send data from the transducer elements 104 back to the processor 116 for processing. Other embodiments may not include the cursor positioning system 108. Ultrasound imaging system 130 may also include a motion sensing system 135 disposed in the scan system 134. The motion sensing system 135 may contain one or more of an accelerometer, an gyro sensor, and a magnetic sensor. The motion sensing system 135 may also be connected to the processor 116. The processor 116 may be able to determine the position and orientation of the scan system 134 based on data from the motion sensing system 135.

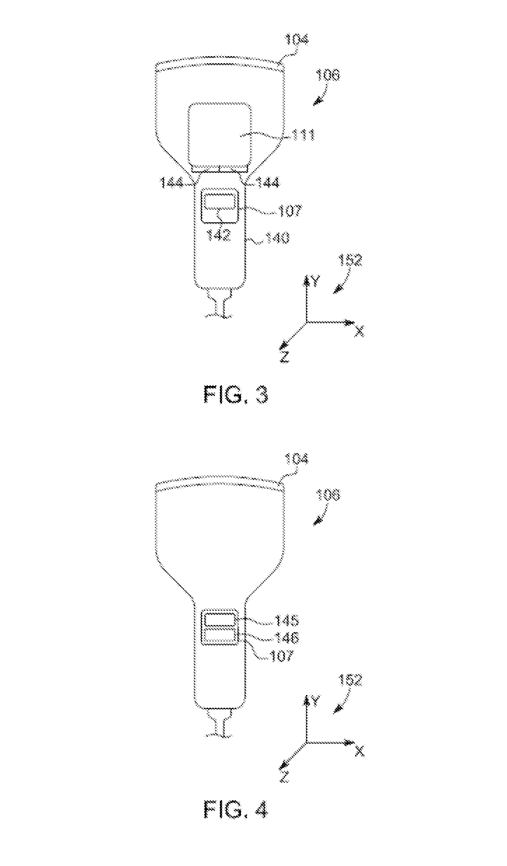

FIGS. 3, 4, and 5 are schematic representations showing additional details of the probe 106 (shown in FIG. 1) in accordance with different embodiments. Common reference numbers will be used to identify identical elements in FIGS. 1, 2, 3, 4, and 5. Structures that were described previously may not be described in detail with respect to FIGS. 3, 4, and 5.

Referring to FIG. 3, the probe 106 includes a housing 140. The motion sensing system 107 includes a magnetic sensor 142. The magnetic sensor 142 will be described in detail hereinafter. According to other embodiments, the motion sensing system 107 may include an accelerometer (not shown) or a gyro sensor (not shown) in place of the magnetic sensor 142. The probe 106 also includes a track pad 111. The track pad 111 may be used to control the position of a cursor on the display device 118 (shown in FIG. 1). For example, the user may use any of their fingers on the track pad 111 to move the cursor. The probe 106 may also optionally include a pair of buttons 144. The pair of buttons 144 may optionally be used to select a location or interact with a graphical user interface (GUI) on the display device 118. The track pad 111 may be positioned elsewhere on the probe 106 in other embodiments. Each one of the pair of buttons 144 may be assigned a different function so that the user may implement either a “left click” or “right click” to access different functionality through the GUI. Other embodiments may not include the pair of buttons 144. Instead, the user may select locations and interact with the GUI through the track pad 111. For example, the user may perform actions such as a “tap” or a “double-tap” on the track pad 111 to access the same functionality that would have otherwise been accessed through the pair of buttons 144.

FIG. 4 is a schematic representation of the probe 106 in accordance with another embodiment. The probe 106 shown in FIG. 4 does not include the track pad 111 and pair of buttons 144 shown in the embodiment of FIG. 3. The motion sensing system 107 of the probe 106 includes both an accelerometer 145 and a gyro sensor 146. The accelerometer 145 and the gyro sensor 146 will be described in additional detail hereinafter. According to other embodiments, the motion sensing system 107 may include any two of the sensors selected from the following group: the gyro sensor 146, the accelerometer 145, and the magnetic sensor (not shown).

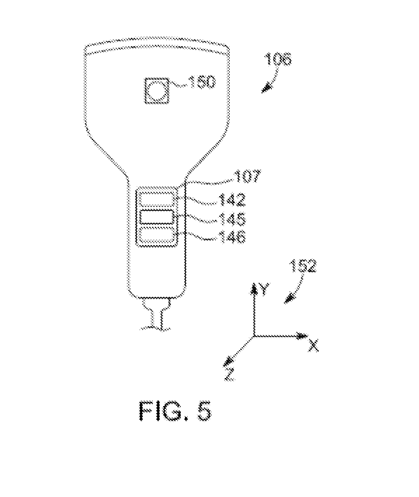

FIG. 5 is a schematic representation of the ultrasound probe 106 in accordance with another embodiment. The probe 106 includes a pointer stick 150 in place of the track pad 111 shown in FIG. 3. The pointer stick 150 may be a rubber-coated joystick that is adapted to control the position of a cursor or reticle on the display device 118. The pointer stick 150 is shown in a location where it may be operated with either the thumb or the forefinger depending on the clinician's grip while using the probe 106. The pointer stick 150 may be positioned elsewhere on the probe 106 in other embodiments due to ergonomic considerations. The motion sensing system 107 of the probe 106 shown in FIG. 5 includes three sensors: the magnetic sensor 142, the accelerometer 145, and the gyro sensor 146. A coordinate system 152 is shown in FIGS. 3, 4, and 5. The coordinate system 152 includes an x-direction, a y-direction and a z-direction. Any two of the directions, or vectors, shown on the coordinate system 152 may be used to define a plane. The coordinate system 152 will be described in additional detail hereinafter.

Referring to FIGS. 3, 4, and 5, the magnetic sensor 142 may include three coils disposed so each coil is mutually orthogonal to the other two coils. For example, a first coil may be disposed in an x-y plane, a second coil maybe disposed in a x-z plane, and a third coil may be disposed in a y-z plane. The coils of the magnetic sensor 142 may be tuned to be sensitive to the strength and direction of a magnetic field that is external to the magnetic sensor 142. For example, the magnet field may be generated by a combination of the earth's magnetic field and/or another magnetic field generator. By detecting magnetic field strength and direction data from each of the three coils in the magnetic sensor 142, the processor 116 (shown in FIG. 1) may be able to determine the absolute position and orientation of the probe 106. According to an exemplary embodiment, the magnetic field generator may include either a permanent magnet or an electromagnet placed externally to the probe 106. For example, the magnetic field generator may be a component of the scan system 101 (shown in FIG. 1).

The accelerometer 145 may be a 3-axis accelerometer, adapted to detect acceleration in any of three orthogonal directions. For example, a first axis of the accelerometer may be disposed in an x-direction, a second axis may be disposed in a y-direction, and a third axis may be disposed in a z-direction. By combining signals from each of the three axes, the accelerometer 145 may be able to detect accelerations in any three-dimensional direction. By integrating accelerations occurring over a period of time, the processor 116 (shown in FIG. 1) may generate an accurate real-time velocity and position of the accelerometer 145, and hence the probe 106, based on data from the accelerometer 145. According to other embodiments, the accelerometer 145 may include any type of device configured to detect acceleration by the measurement of force in specific directions.

The gyro sensor 146 is configured to detect changes angular velocities and changes in angular momentum, and it may be used to determine angular position information of the probe 106. The gyro sensor 146 may detect rotations about any arbitrary axis. The gyro sensor 146 may by a vibration gyro, a fiber optic gyro, or any other type of sensor adapted to detect rotation or change in angular momentum.

Referring now to FIGS. 1, 4, and 5, the combination of data from the gyro sensor 146 and the accelerometer 145 may be used by the processor 116 for calculating the position, orientation, and velocity of the probe 106 without the need for an external reference. According to other embodiments, a processor used for calculating the position, orientation, and velocity may be located in the probe 106. The motion sensing system 107 may be used to detect many different types of motion. For example, the motion sensing system 107 may be used to detect translations, such as moving the probe 106 up and down (also referred to as heaving), moving the probe left and right (also referred to as swaying), and moving the probe 106 forward and backward (also referred to as surging). Additionally, the motion sensing system 107 may be used to detect rotations, such as tilting the probe 106 forward and backward (also referred to as pitching), turning the probe 106 left and right (also referred to as yawing), and tilting the probe 106 from side to side (also referred to as rolling).

When a user performs or “draws” a gesture in 3D space with the probe 106, the processor 116 may convert data from the motion sensing system 107 into linear and angular velocity signals. Next, the processor 116 may convert the 3D gestures into 2D movements. The processor 116 may use these 2D movements as inputs for performing gesture recognition.

By tracking the linear acceleration with an accelerometer 145, the processor 116 may calculate the linear acceleration of the probe 106 in an inertial reference frame. Performing an integration on the inertial accelerations and using the original velocity as the initial condition, enables the processor 116 to calculate the inertial velocities of the probe 106. Performing an additional integration and using the original position as the initial condition allows the processor 116 to calculate the inertial position of the probe 106. The processor 116 may also measure the angular velocities and angular acceleration of the probe 106 using the data from the gyro sensor 146. The processor 116 may, for example, use the original orientation of the probe 106 as an initial condition and integrate the changes in angular velocity, as measured by the gyro sensor 146, to calculate the probe's 106 angular velocity and angular position at any specific time. With regularly sampled data from the accelerometer 145 and the gyro sensor 146, the processor 116 may compute the position and orientation of the probe 106 at any time.

The exemplary embodiment of the probe 106 shown in FIG. 5 is particularly accurate for tracking the position and orientation of the probe 106 due to the synergy between the attributes of the different sensor types. For example, the accelerometer 145 is capable of detecting translations of the probe 106 with a high degree of precision. However, the accelerometer 145 is not well-suited for detecting angular rotations of the probe 106. The gyro sensor 146, meanwhile, is extremely well-suited for detecting the angle of the probe 106 and/or detecting changes in angular momentum resulting from rotating the probe 106 in any arbitrary direction. Pairing the accelerometer 145 with the gyro sensor 146 is appropriate because together, they are adapted to provide very precise information on both the translation of the probe 106 and the orientation of the probe 106. However, one drawback of both the accelerometer 145 and the gyro sensor 146 is that both sensor types are prone to “drift” over time. Drift refers to intrinsic error in a measurement over time. The magnetic sensor 142 allows for the detection of an absolute location in space with better accuracy than just the combination of the accelerometer 144 and the gyro sensor 146. Even though the position information from the magnetic sensor 142 may be relatively low in precision, the data from the magnetic sensor 142 may be used to correct for systematic drifts present in the data measured by one or both of the accelerometer 144 and the gyro sensor 146. Each of the sensor types in probe 106 shown in FIG. 5 has a unique set of strengths and weaknesses. However, by packaging all three sensor types in the probe 106, the position and orientation of the probe 106 may be determined with enhanced accuracy and precision.

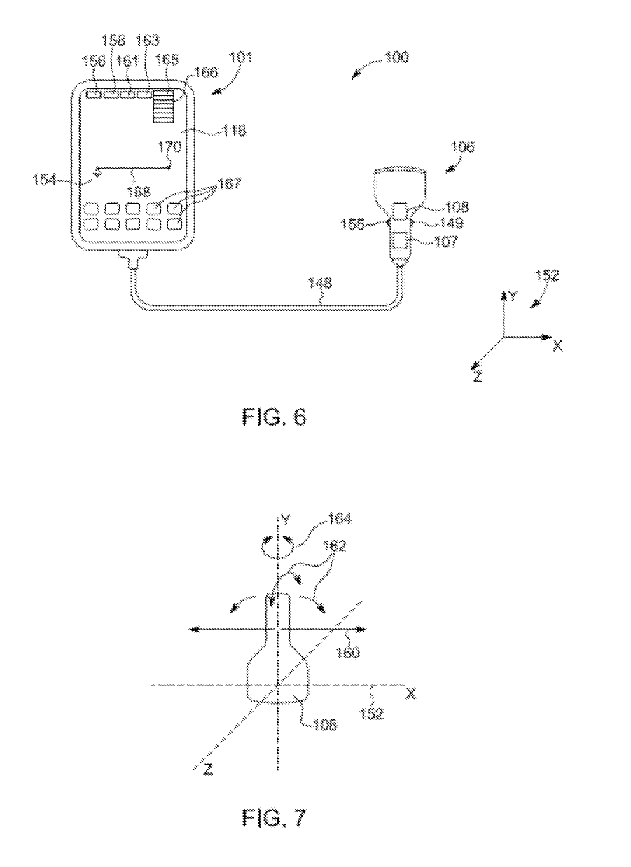

FIG. 6 is a schematic representation of a hand-held or hand-carried ultrasound imaging system 100 in accordance with an embodiment. Ultrasound imaging system 100 includes the scan system 101 and the probe 106 connected by a cable 148 in accordance with an embodiment. According to other embodiments, the probe 106 may be in wireless communication with the scan system 101. The probe 106 includes the motion sensing system 107. The motion sensing system 107 may, for example, be in accordance with any of the embodiments described with respect to FIG. 3, 4 or 5. The probe 106 may also include the cursor positioning device 108 and a first switch 149. The probe 106 may not include one or both of the cursor positioning device 108 and the first switch 149 in accordance with other embodiments. The scan system 101 includes the display device 118, that may include an LCD screen, an LED screen, or other type of display. Coordinate system 152 includes three vectors indicating an x-direction, a y-direction, and a z-direction. The coordinates system 152 may be defined with respect to the room. For example, the y-direction may be defined as vertical and the x-direction may be defined as being with respect to a first compass direction while the z-axis may be defined with respect to a second compass direction. The orientation of the coordinate system 152 may be defined with respect to the scan system 101 according to other embodiments. For example, according to an exemplary embodiment, the orientation of the coordinate system 152 may be adjusted in real-time so that it is always in the same relationship with respect to the display device 118. According to one embodiment, the x-y plane, defined by the x-direction and the y-direction of the coordinate system 152 may always be oriented so that it is parallel to a viewing surface of the display device 118. According to other embodiments, the clinician may manually set the orientation of the coordinate system 152.

FIG. 7 is a schematic representation of the probe 106 overlaid on a Cartesian coordinate system 152. The motion sensing system 107 (shown in FIG. 6) may detect the position and orientation of the probe 106 in real-time in accordance with an embodiment. Based on data from the motion sensing system 107, the processor 116 (shown in FIG. 1) may determine exactly how the probe 106 has been manipulated. Based on the data from the motion sensing system 107, the processor 116 may also detect any number of gestures, or specific patterns of movement, performed by the clinician with the probe 106. The probe 106 may be translated, as indicated by path 160, the probe 106 may be tilted as indicated by paths 162, and the probe may be rotated as indicated by path 164. It should be appreciated by those skilled in the art that the paths 160, 162, and 164 represent a limited subset of all the gestures which may be performed with the probe 106 and detected with the motion sensing system 107. By combining data from the motion sensing system 107 to identifying translations, tilt, and rotations, the processor 116 may detect any gesture performed with the probe 106 in three-dimensional space.

Referring to FIG. 6, gestures performed with the probe 106 may be used for a variety of purposes including performing a control operation. It may be necessary to first input a command to select or activate a specific mode. For example, when activated, the mode may use gestures performed with the probe 106 to interface with a graphical user interface (GUI) and/or control the position of a cursor 154 or reticle on the display device 118. According to an embodiment, the clinician may input the command to activate a particular mode by performing a very specific gesture that is unlikely to be accidentally performed during the process of handling the probe 106 or scanning a patient. A non-limiting list of gestures that may be used to select the mode includes moving the probe 106 in a back-and-forth motion or performing a flicking motion with the probe 106. According to other embodiments, the clinician may select a control or switch on the probe 106, such as a second switch 155, in order to toggle between different modes. The clinician may also select a hard or soft key or other user interface device on the scan system 101 to control the mode of the ultrasound imaging system 100.

According to other embodiments, the processor 116 may be configured to perform multiple control operations in response to a single gesture performed with the probe 106. For example, the processor 116 may perform a series of control operations that are all part of a script, or sequence of commands. The script may include multiple control operations that are commonly performed in a sequence, or the script may include multiple control operations that need to be performed in a sequence as part of a specific procedure. For example, the processor 116 may be configured to detect a gesture and then perform both a control operation and a second control operation in response to the gesture. Additionally, according to other embodiments, a single gesture may be associated with two or more different control operations depending upon the mode of operation of the ultrasound imaging system 100. A gesture may be associated with a first control operation in a first mode of operation and the same gesture may be associated with a second control operation in a second mode of operation. For example, a gesture may be associated with a control operation such as “scan” in a first mode of operation, while the same gesture may be associated with a second control operation such as “archive” or “freeze” in a second mode of operation. It should be appreciated that a single gesture could be associated with many different control operations depending on the mode of operation.

The ultrasound imaging system 100 may also be configured to allow the clinician to customize one or more of the gestures used to input a command. For example, the user may first select a command in order to configure the system to enable the learning of a user-defined gesture. According to an embodiment, the user-defined gesture may include any pattern or motion performed by the user with the probe 106. For purposes of this disclosure, this mode of the ultrasound imaging system 100 will be referred to as a learning mode. The user may then perform the user-defined gesture at least once while in the learning mode. The user may want to perform the user-defined gesture multiple times in order to increase the robustness of the processor's 116 ability to accurately identify the gesture based on the data from the motion sensing system 107. For example, by performing the user-defined gesture multiple times, the processor 116 may establish both a baseline for the user-defined gesture as well as a statistical standard of deviation for patterns of motion that should still be interpreted as the intended gesture. The clinician may then associate the user-defined gesture with a specific control operation, such as a function or a command for the ultrasound imaging system 100.

The clinician may, for example, use gestures to interface with a GUI. The position of a graphical indicator, such as cursor 154, may be controlled with gestures performed with the probe 106. According to an exemplary embodiment, the clinician may translate the probe 106 generally in x and y directions and the processor 116 may adjust the position of the cursor 154 in real-time in response to the x-y position of the probe 106. In other words: moving the probe 106 to the right would result in cursor 154 movement to the right; moving the probe 106 to the left would result in cursor 154 movement to the left; moving the probe 106 up would result in cursor 154 movement to in the positive y direction; and moving the probe 106 down would result in cursor 154 movement in the negative y-direction. According to an exemplary embodiment, probe 106 movements in the z-direction may not affect the position of the cursor 154 on the display device 118. It should be appreciated that this represents only one particular mapping of probe gestures to cursor 154 position.

In other embodiments, the position of the probe 106 may be determined relative to a plane other than the x-y plane. For example, it may be more ergonomic for the clinician to move the probe relative to a plane that is tilted somewhat from the x-y plane. Additionally, in other embodiments, it may be easier to determine the cursor position based the probe 106 position with respect to the x-z plane or the y-z plane.

The clinician may be able to select the desired plane in which to track probe movements. For example, the clinician may be able to adjust the tilt and angle of the plane through the user interface on the scan system 101. As described previously, the clinician may also be able to define the orientation of coordinate system 152. For example, the position of the probe 106 when the “cursor control” mode is selected may determine the orientation of the coordinate system 152. According to another embodiment, the scan system 101 may also include a motion sensing system, similar to the motion sensing system 107 described with respect to the probe 106. The processor 116 may automatically orient the coordinate system 152 so that the X-Y axis of the coordinate axis is positioned parallel to a display surface of the display device 118. This provides a very intuitive interface for the clinician, since it would be natural to move the probe 106 in a plane generally parallel to the display surface of the display device 118 in order to reposition the cursor 154.

According to another embodiment, it may be desirable to control zoom with gestures from the probe 106 at the same time as the cursor 154 position. According to the exemplary embodiment described above, the position of the cursor 154 may be controlled based on the real-time position of the probe 106 relative to the x-y plane. The zoom may be controlled based on the gestures of the probe 106 with respect to the z-direction at the same time. For example, the clinician may zoom in on the image by moving the probe further away from the clinician in the z-direction and the clinician may zoom out by moving the probe 106 closer to the clinician in the z-direction. According to other embodiments, the gestures controlling the zoom-in and zoom-out functions may be reversed. By performing gestures with the probe 106 in 3D space, the user may therefore simultaneously control both the zoom of the image displayed on the display device 118 and the position of the cursor 154.

Still referring to FIG. 6, an example of a GUI is shown on the display device 118. The GUI includes a first menu 156, a second menu 158, a third menu 161, a fourth menu 163, and a fifth menu 165. A dropdown menu 166 is shown cascading down from the fifth menu 165. The GUI also includes a plurality of soft keys 167, or icons, each controlling an image parameter, a scan function, or another selectable feature. According to an embodiment, the clinician may position the cursor 154 on any portion of the display device 118. The clinician may select a menu 156, 158, 161, 163, and 165 or any of the plurality of soft keys 167. For example, the clinician could select one of the menus, such as the fifth menu 165, in order to make the dropdown menu 166 appear.

According to an embodiment, the user may control the cursor 154 position based on gestures performed with the probe 106. The clinician may position the cursor 154 on the desired portion of the display device 118 and then select the desired soft key 167 or icon. It may be desirable to determine measurements or other quantitative values based on ultrasound data. For many of these measurements or quantitative values it is necessary for a user to select one or more points on the image so that the appropriate value may be determined. Measurements are common for prenatal imaging and cardiac imaging. Typical measurements include head circumference, femur length, longitudinal myocardial displacement, ejection fraction, and left ventricle volume just to name a few. The clinician may select one or more points on the image in order for the processor 116 to calculate the measurement. For example, a first point 170 is shown on the display device 118. Some measurements may be performed with only a single point, such as determining a Doppler velocity or other value associated with a particular point or location. A line 168 is shown connecting the first point 170 to the cursor 154. According to an exemplary workflow, the user may first position the cursor 154 at the location of the first point 170 and select that location. Next, the user may position the cursor at a new location, such as where the cursor 154 is shown in FIG. 6. The user may then select a second point (not shown) that the processor 116 would use to calculate a measurement. According to one embodiment, the clinician may select an icon or select a measurement mode with a control on the probe 106, such as second switch 155. Or, the clinician may perform a specific gesture with the probe 106 to select an icon or place one or more points that will be used in a measurement mode. The clinician may, for example, move the probe 106 quickly back-and-forth to select an icon or select a point. Moving the probe 106 back-and forth a single time may have same effect as a single click with a mouse. According to an embodiment, the clinician may move the probe 106 back-and forth two times to have the same effect as a double-click with a mouse. According to another exemplary embodiment, the clinician may select an icon or select a point by performing a flicking motion with the probe 106. The flicking motion may, for instance, include a relatively rapid rotation in a first direction and then a rotation back in the opposite direction. The user may perform either the back-and-forth motion or the flicking motion relatively quickly. For example, the user may complete the back-and-forth gesture or the flicking motion within 0.5 seconds or less according to an exemplary embodiment. Other gestures performed with the probe 106 may also be used to select an icon, interact with the GUI, or select a point according to other embodiments.

According to other embodiments, the user may control the position of the cursor 154 with the cursor positioning device 108. As described previously, the cursor positioning device 108 may include a track pad 111 or a pointer stick 150 according to embodiments. The clinician may use the cursor positioning device 108 to position the cursor 154 on display device 118. For example, the clinician may guide the cursor 154 with either a finger, such as a thumb or index finger, to the desired location on the display device 118. The clinician may then either select a menu, interact with the GUI or establish one or more points for a measurement using the cursor positioning device 108.

Referring to FIG. 1, the motion sensing system 107 in the probe 106 may also be used to collect position data during the acquisition of ultrasound data. For example, position data collected by the motion sensing system 107 may be used to reconstruct three-dimensional (3D) volumes of data acquired during a free-hand scanning mode. During the free-hand scanning mode, the operator moves the probe 106 in order to acquire data of a plurality of 2D planes. For purposes of this disclosure, data acquired from each of the planes may be referred to as a “frame” of data. The term “frame” may also be used to refer to an image generated from data from a single plane. By using the position data from the motion sensing system 107, the processor 116 is able to determine the relative position and orientation of each frame. Then using the position data associated with each frame, the processor 116 may reconstruct a 3D volume by combining a plurality of frames. The addition of the motion sensing system 107 to the probe 106 allows the clinician to acquire volumetric data with a relatively inexpensive probe 106 without requiring a mechanical sweeping mechanism or full beam-steering in both azimuth and elevation directions.

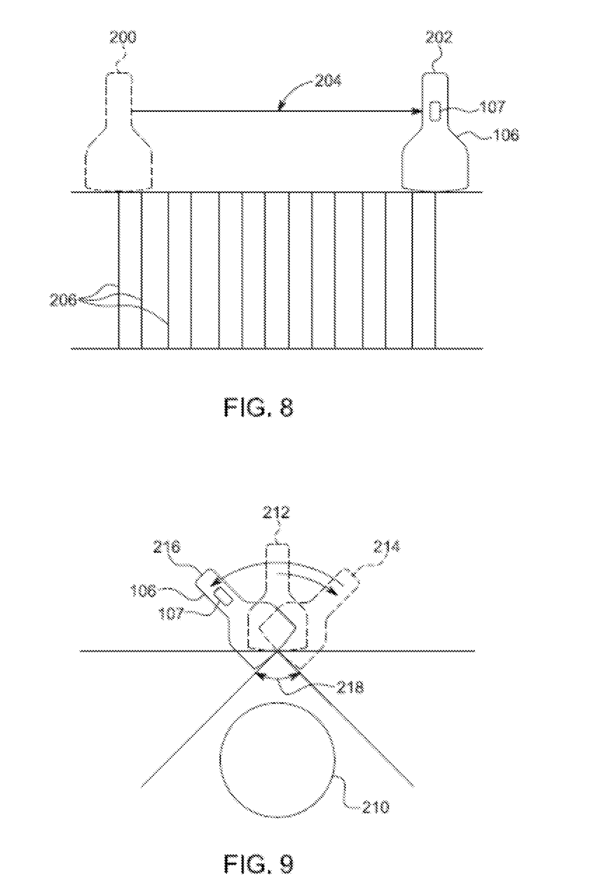

FIG. 8 is schematic representation of a scan acquisition pattern in accordance with an embodiment. The scan acquisition pattern shown in FIG. 8 is a linear translation. The probe 106 is translated from first position 200 to second position 202 along a path 204. The initial position of the probe 106 is indicated by a dashed outline of the probe 106. The exemplary path 204 is generally linear, but it should be appreciated that the translation path may not be linear in other embodiments. For example, the clinician would typically scan along the surface of the patient's skin. The translation path will therefore typically follow the contours of the patient's anatomy being scanned. Multiple 2D frames of data are acquired of planes 206. The planes 206 are shown from side perspective so that they appear as lines in FIG. 8. The motion sensing system 107 detects the position and orientation of each plane 206 while acquiring the ultrasound data. As described earlier, the processor 116 uses these data when reconstructing a 3D volume based on the 2D frames of data. By knowing the exact relationship between each of the acquired planes 206, the processor 116 may generate and reconstruct a more accurate volumetric, or 3D, dataset.

In addition to translation, other acquisition patterns may be used when acquiring ultrasound data. FIG. 9 shows a schematic representation of a scan acquisition pattern that may also be used to acquire 3D, or volumetric, data. FIG. 9 shows an embodiment where the probe 106 is tilted though an angle in order to acquire a volume of data. According to an exemplary embodiment shown in FIG. 9, the probe 106 is tilted from first position 212 in a first direction to second position 214. Next, the clinician tilts the probe 106 from second position 214 to third position 216 in a second direction that is generally opposite of the first direction. In the process of tilting the probe 106, the clinician causes the probe to sweep through an angle 218, thereby acquiring volumetric data of bladder 210. The bladder 210 is just one exemplary portion of anatomy that could be scanned. It should be appreciated that other anatomical structures may be scanned in accordance with other embodiments. As with the linear translation described above, data from the motion sensing system 107 may be used to identify the positions of all the frames that are acquired while tilting the probe through angle 218.

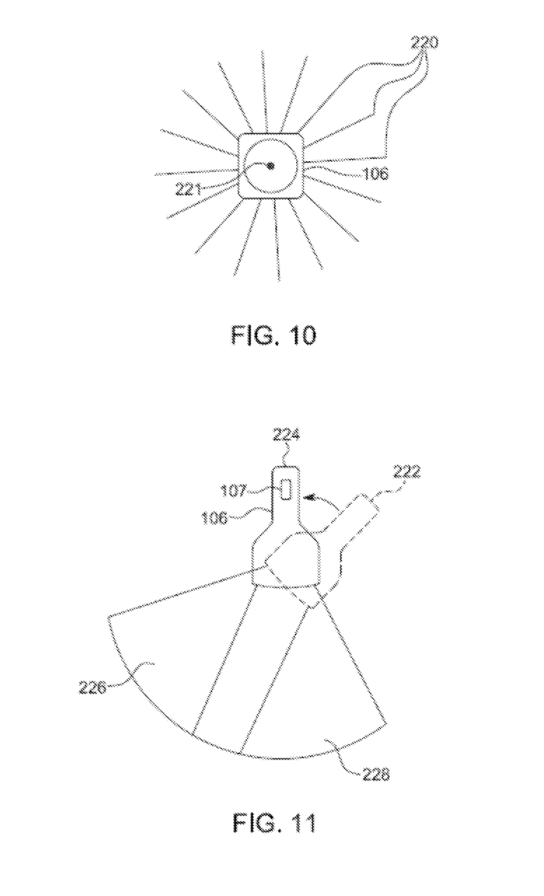

FIG. 10 is a schematic representation of a scan acquisition pattern in accordance with an embodiment. FIG. 10 shows the probe 106 in a top view. According to an embodiment, a volume acquisition may also be performed by rotating the probe through approximately 180 degrees. Ultrasound data from a plurality of planes 220 are acquired while the clinician rotates the probe 106. As described previously, the motion sensing system 107 (shown in FIG. 6) may collect position data during the process of acquiring ultrasound data while rotating the probe 106. The processor 116 (shown in FIG. 1) may then use the position data to reconstruct volumetric data from the frames of data of the planes 220.

FIG. 11 is a schematic representation of a scan acquisition pattern in accordance with an embodiment. The scan acquisition pattern involves tilting the probe 106 in a direction generally parallel to the imaging plane. In the embodiment shown in FIG. 11, the probe 106 is tilted from a first position 222 to a second position 224. The first position 222 of the probe 106 is indicated by the dashed line. In the process of tilting the probe 106, a first frame of data 226 is acquired from the first position 222 and a second frame of data 228 is acquired form the second or final position 224. By using the data from the motion sensing system 107, the processor 116 may combine the first frame of data 226 and the second frame of data 228 to create a panoramic image with a wider field of view since the first frame of data 226 and the second frame of data 228 are generally coplanar.

According to an embodiment, data from the motion sensing system 107 may be used to detect a type of scan or to automatically start and stop the acquisition of ultrasound data for a volume. Additionally, the probe 106 may automatically come out of a sleep mode when motion is detected with the motion sensing system. The sleep mode, may, for instance, be a mode where the transducer elements are not energized. As soon as movement is detected, the transducer elements may begin to transmit ultrasound energy. After the probe 106 has been stationary for a predetermined amount of time, the processor 116, or an additional processor on the probe 106 (not shown) may automatically cause the probe 106 to return to a sleep mode. By toggling between a sleep mode when the probe 106 is not being used for scanning and an active scanning mode, it is easier to maintain lower probe 106 temperatures and conserve power.

Referring to FIG. 8, the processor 116 (shown in FIG. 1) may use data from the motion sensing system 107 to determine that the probe 106 has been translated along the surface of a patient. The processor may detect the when the probe 106 is first translated from first position 200 and when the probe 106 is no longer being translated at second position 202. According to an embodiment, ultrasound data is temporarily stored in the memory 120 (shown in FIG. 1) during the acquisition process. By detecting the start and the finish of movement corresponding to the acquisition of data for a volume, the processor 116 may associate the appropriate data with the volume acquisition. This may include associating a position and orientation for each frame of data. Referring to FIG. 8, all the frames of data acquired from planes 206 between first position 200 and second position 202 may be used to generate the volumetric data.

FIG. 9 shows a schematic representation of an embodiment where the user acquires volumetric data by tilting the probe 106 through a range of degrees, from a first position 212, to a second position 214, and then to a third position 216. FIG. 9 will be described in accordance with an embodiment where the user is acquiring volumetric data of a bladder. It should be appreciated that acquiring data of a bladder is just one exemplary embodiment and that volumetric data of other structures may be acquired by tilting the probe 106 in the manner similar to that represented in FIG. 9.

Still referring to FIG. 9, the clinician initially positions the probe 106 at a position, where he or she can clearly see a live 2D image of the bladder 210 displayed on the display device 118 (shown in FIG. 6). The clinician may adjust the position of the probe 106 so that the live 2D image is in approximately the center of the bladder 210, such as when the probe 106 is positioned at first position 212. Next the user tips the probe 106 in a first direction from first position 212 to second position 214. The clinician may tilt the probe 106 until the bladder is no longer visible on the live 2D image displayed on the display device 118 in order to ensure that the probe 106 has been tipped a sufficient amount. Next, the clinician may tip the probe 106 in a second direction, generally opposite to the first direction, towards third position 216. As before, the clinician my view the live 2D image while tipping the probe 106 in the second direction to ensure that all of the bladder 210 has been captured.

The processor 116 may identify the gesture, or pattern of motion, performed with the probe 106 in order to capture the volumetric data. The volumetric data may include data of the bladder 210. The processor 116 may automatically tag each of the 2D frames of data in a buffer or memory as part of a volume in response to detecting a tilt in a first direction followed by a tilt in the second direction. In additional, position and orientation data collected from the motion sensing system 107 may be associated with each of the frames. While the embodiment represented in FIG. 9 describes tilting the probe 106 in a first direction and then in a second direction to acquire volumetric data, it should be appreciated that the according to other embodiments, the user could acquire volumetric data by simply tilting the probe through the angle 218 in a single motion if the location of the target anatomy were already known.

FIG. 10 shows a schematic representation of an acquisition pattern for acquiring volumetric data. The acquisition patter represented in FIG. 10 involves rotating the probe 106 about a longitudinal axis 221 in order to acquire 2D data along a plurality of planes 220. The processor 116 (shown in FIG. 1) may use data from the motion sensing system 107 (shown in FIG. 1) to determine when the probe 106 has been rotated a sufficient amount in order to generate volumetric data. According to an embodiment, it may be necessary to rotate the probe 106 though at least 180 degrees in order to acquire complete volumetric data for a given volume. The processor 116 may associate the data stored in the memory 120 (shown in FIG. 1) with position and orientation data from the motion sensing system 107. The processor may then use the position and orientation data of each of the planes 220 to generate volumetric data.

FIG. 11 shows a schematic representation of a gesture, or an acquisition pattern, for acquiring an image with an extended field of view. According to the embodiment shown in FIG. 11, the user tilts the probe 106 from the first position 222 to a second position 224. The user acquires a first frame of data 226 at the first position 222 and a second frame of data 228 at the second position 224. The probe 106 is tilted in a direction that is generally parallel to the first frame of data 226, thus allowing the clinician to acquire data of a larger field-of-view. The processor 116 (shown in FIG. 1) may receive data from the motion sensing system 107 indicating that the probe 106 has been tilted in a direction that is generally parallel to the first frame 226. In response to receiving this data from the motion sensing system 107, the processor 116 may identify the motion as belonging to an acquisition for an extended field-of-view and the processor 116 may automatically combine the data from the first frame 226 with the data from the second frame 228 in order to generate and display a panoramic image with an extended field-of-view.

The processor 116 may automatically display a rendering of the volumetric data after detecting that a volume of data has been acquired according to any of the embodiments described with respect to FIGS. 8, 9, and 10. Additionally, the processor 116 may cause the ultrasound imaging system to display some kind of cue once a complete set of volumetric data has been successfully acquired according to any of the previously described embodiments. For example, the processor 116 may control the generation of an audible cue, or the processor 116 may display a visual cue on the display device 118 (shown in FIG. 6).

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of controlling an ultrasound imaging system, the method comprising:
performing a gesture with a probe;
detecting the gesture based on data from a motion sensing system in the probe, wherein the motion sensing system includes at least one sensor selected from the group consisting of an accelerometer, a gyro sensor, and a magnetic sensor; and
performing a control operation based on the detected gesture.

2. The method of claim 1, wherein said performing the gesture comprises translating the probe and the control operation comprises repositioning a graphical indicator in response to said translating the probe.

3. The method of claim 1, wherein said performing the gesture comprising performing a flicking motion with the probe and the control operation comprises selecting a function in response to performing the flicking motion.

4. The method of claim 1, wherein said performing the gesture comprises moving the probe in a back-and-forth motion and the control operation comprises selecting a function in response to moving the probe in a back-and-forth motion.

5. The method of claim 1, wherein the control operation comprises a measurement.

6. The method of claim 1, further comprising inputting a command through a cursor positioning device on the probe and implementing an action based on the command.

7. The method of claim 6, wherein said inputting the command comprises inputting the command through either a touch screen on the probe or through a pointer stick on the probe.

8. The method of claim 1, wherein the control operation comprises interfacing with a graphical user interface on a display device.

9. A method of controlling an ultrasound imaging system, the method comprising:
inputting a command to select a measurement mode;
displaying a graphical indicator on a display device;
performing a gesture with a probe;
detecting the gesture based on data from a motion sensing system in the probe, wherein the motion sensing system includes at least one sensor selected from a group consisting of an accelerometer, a gyro sensor, and a magnetic sensor;
repositioning the graphical indicator based on the detected gesture;
selecting a position indicated by the graphical indicator after said repositioning the graphical indicator; and
performing a measurement using the selected position.

10. The method of claim 9, wherein said inputting the command to select the measurement mode comprises performing a second gesture with the probe that is different from the gesture.

11. The method of claim 9, wherein said inputting the command to select the measurement mode comprises activating a control on the probe.

12. The method of claim 9, wherein said selecting the position comprises performing a second gesture with the probe that is different from the gesture.

13. An ultrasound imaging system comprising:
a probe, the probe comprising:
a housing;
at least one transducer element disposed in the housing; and
a motion sensing system either attached to the housing or disposed in the housing; and
a scan system in communication with the probe, the scan system comprising:
a display device; and
a processor, wherein the processor is configured to receive data from the motion sensing system and to interpret the data as a gesture, and wherein the processor is configured to perform a control operation based on the gesture.

14. The ultrasound imaging system of claim 13, wherein the motion sensing system comprises at least one sensor selected from the group consisting of a magnetic sensor, an accelerometer, and a gyro sensor.

15. The ultrasound imaging system of claim 13, wherein the motion sensing system comprises an accelerometer and a gyro sensor.

16. The ultrasound imaging system of claim 13, wherein the probe further comprises a control and the control is configured to toggle between an imaging mode and a measurement mode.

17. The ultrasound imaging system of claim 13, wherein the probe further comprises a cursor-positioning device mounted to the housing, and wherein the cursor-positioning device is configured to control the position of a graphical indicator displayed on the display device.

18. The ultrasound imaging system of claim 17, wherein the cursor-positioning device comprises a track pad.

19. The ultrasound imaging system of claim 17, wherein the ultrasound imaging system comprises a hand-held ultrasound imaging system.

20. The ultrasound imaging system of claim 13, wherein the processor is further configured with a learning mode to associate a user-defined gesture with a specific control operation.

21. The ultrasound imaging system of claim 13, wherein the processor is further configured to perform a second control operation based on the gesture after performing the control operation, and wherein the control operation and the second control operation are part of a script.

22. The ultrasound imaging system of claim 13, wherein the processor is configured to perform the control operation based on the gesture when in a first mode of operation and wherein the processor is configured to perform a second control operation based on the gesture when in a second mode of operation.
